(19) United States
(12) Patent Application Publication
(10) Pub. No.: US 2019/0169654 A1
(43) Pub. Date: Jun. 6, 2019

(54) PROCESS FOR PRODUCING ALCOHOLS UNDER AEROBIC CONDITIONS AND PRODUCT EXTRACTION USING OLEYL ALCOHOL

(71) Applicant: Evonik Degussa GmbH, Essen (DE)

(72) Inventors: Anja HECKER, Muenster (DE); Thomas HAAS, Muenster (DE); Simon BECK, Muenster (DE)

(73) Assignee: Evonik Degussa GmbH, Essen (DE)

(21) Appl. No.: 16/320,836

(22) PCT Filed: Jul. 25, 2017
(86) PCT No.: PCT/EP2017/068783

§ 371 Date: Jan. 25, 2019

(30) Foreign Application Priority Data
Jul. 27, 2016 (EP) 16181432.2

(51) Int. Cl. C12P 7/04; C07C 29/86; C12P 39/00; C12P 7/16

(52) U.S. Cl. CPC C12P 7/04; C12P 7/16; C12P 39/00; C07C 29/86

(57) ABSTRACT

The present invention relates to a method of producing at least one alcohol from a carbon source, the method comprising: (a) producing the alcohol in an aqueous medium under aerobic conditions; and (b) extracting the alcohol from step (a) from the aqueous medium by: (bi) contacting the alcohol in the aqueous medium with at least one extracting medium for a time sufficient to extract the alcohol from the aqueous medium into the extracting medium, (bii) separating the extracting medium with the extracted alcohol from the aqueous medium wherein the extracting medium comprises:—oleyl alcohol; or—polypropylene glycol and an alkane wherein the alcohol comprises at least 3 carbon atoms.

### FIELD OF THE INVENTION

The present invention relates to a biotechnological method of producing alcohols including higher alcohols from a carbon source in aerobic conditions. In particular, the method relates to a biotechnological production of at least one alcohol in an aqueous solution in the presence of oxygen and a means of extracting the alcohol from the aqueous solution.

### BACKGROUND OF THE INVENTION

Biotechnological methods of producing alcohols, particularly ethanol are well known in the art. Especially the use of acetogenic bacteria on various carbon sources to produce ethanol and/or acetate is well known. However, in most cases, the production of alcohols can only be successfully carried out in the absence of oxygen. This phenomenon is confirmed at least by Brioukhanov, 2006, Imlay, 2006, Lan, 2013 and the like where it is shown that acetogenic bacteria do not successfully produce ethanol in aerobic conditions. Therefore, in the current methods known in the art, carbon substrates comprising oxygen, such as waste gases from steel mills are first processed to remove the oxygen before they are introduced to the acetogenic cells for ethanol and/or acetate production. The oxygen separation step makes the process more expensive and time consuming. Further, there may be some loss in the raw materials during this step of separation.

There is thus a need in the art for a means of producing ethanol and/or acetate in the presence of oxygen. Ethanol may then be used as a raw material for production of higher carbon compounds such as alcohols, acids and the like.

For example, butanol and higher alcohols have several uses including being used as fuel. For example, butanol in the future can replace gasoline as the energy contents of the two fuels are nearly the same. Further, butanol has several other superior properties as an alternative fuel when compared to ethanol. These include butanol having higher energy content, butanol being less “evaporative” than ethanol or gasoline and butanol being easily transportable compared to ethanol. For these reasons and more, there is already an existing potential market for butanol and/or related higher alcohols. Butanol and other higher alcohols are also used as industrial solvents.

Similarly, propanol is a solvent used in the pharmaceutical industry for resins and cellulose esters amongst other compounds. This solvent, which is better known as isopropanol or isopropyl alcohol, is widely used on printing ink and in the printing industry. 1-propanol is produced in nature by the decomposition of organic materials by a variety of microorganisms and may be found in plants and fusel oil. 1-propanol can also be produced from petrochemically-derived ethene by a reaction with carbon monoxide and hydrogen to give propionaldehyde, which is then hydrogenated. It is also a by-product of methanol manufacture and may be produced from propane directly or from acrolein. Propanol has further other potential uses.

All these alcohols are currently being produced by cracking gasoline or petroleum which is bad for the environment. Further, most of these alcohols produced commercially are being extracted by conventional procedures such as distillation. Distillation of alcohol using these conventional means may require more input of energy than the output energy capability of the extracted alcohol.

Accordingly, there is a need for an environmentally friendly and bio-based efficient alternative to the petro-based production process of alcohols and an energy-efficient way of extracting these bio-based alcohols.

### DESCRIPTION OF THE INVENTION

The present invention provides a biotechnological means of producing at least one alcohol from a carbon source in aerobic conditions. The carbon source may comprise carbon dioxide and/or carbon monoxide. In particular, the method comprises at least two parts. One part that involves the formation of the alcohol in an aqueous medium from the carbon source and a further part which involves the extraction of the alcohol from the aqueous medium.

In one aspect of the present invention, there is provided a method of producing at least one alcohol from a carbon source, the method comprising:
(a) producing the alcohol in an aqueous medium under aerobic conditions; and
(b) extracting the alcohol from step (a) from the aqueous medium by:
   (bi) contacting the alcohol in the aqueous medium with at least one extracting medium for a time sufficient to extract the alcohol from the aqueous medium into the extracting medium,
   (bii) separating the extracting medium with the extracted alcohol from the aqueous medium
   wherein the extracting medium comprises:
      oleyl alcohol; or
      polypropylene glycol and alkane
   wherein the alcohol comprises at least 3 carbon atoms.

The method according to any aspect of the present invention may be an aerobic method of producing at least one isolated alcohol from a carbon source. An isolated alcohol may refer to at least one alcohol that may be separated from the medium where the alcohol has been produced. In one example, the alcohol may be produced in an aqueous medium (e.g. fermentation medium where the alcohol is produced by specific cells from a carbon source). The isolated alcohol may refer to the alcohol extracted from the aqueous medium. In particular, the extracting step allows for the separation of excess water from the aqueous medium thus resulting in a formation of a mixture containing the extracted alcohol.

The extracting medium may also be referred to as the ‘extraction medium’. The extraction medium may be used for extracting/isolating the alcohol produced according to any method of the present invention from the aqueous medium wherein the alcohol was originally produced. At the end of the extracted step, excess water from the aqueous medium may be removed thus resulting in the extracting medium containing the extracted alcohol. The extracting medium may comprise a combination of compounds that may result in an efficient means of extracting the alcohol from the aqueous medium. In particular, the extracting medium may comprise: (i) at least one hydrocarbon comprising at least 5-18 carbon atoms, and (ii) at least one polyoxyalkylene polymer. The extraction medium according to any aspect of the present invention may efficiently extract the fermentation alcohol into the hydrocarbon-polyoxyalkylene polymer extracting medium. In particular, the hydrocarbon in the mixture comprising hydrocarbon-polyoxyalkylene polymer may be least one alkane. More in particular, the alkane may comprise at least 5 carbon atoms. This extracting medium of a mixture of polypropylene glycol and at least one alkane may be considered suitable in the method according to any aspect of the present invention as the mixture works efficiently in extracting the desired alcohol in the presence of oxygen. In particular, the mixture of polypropylene glycol and at least one alkane may be considered to work better than oleyl alcohol as an extracting medium as the double bond in oleyl alcohol may be considered to be sensitive to the presence of oxygen.

Even more in particular, the alkane may comprise at least 5 to 18 carbon atoms. In one example, the alkane may be selected from the group consisting of pentane, hexane, heptane, octane, nonane, decane, undecane, dodecane, tridecane, tetradecane, pentadecane, hexadecane, heptadecane and octadecane. In a further example, the extracting medium may comprise a mixture of polypropylene glycol and hexadecane.

More in particular, the mixture of polypropylene glycol and hexadecane may comprise about 50, 55, 60, 65, 70, 75, 80% w/w of polypropylene glycol and 50, 45, 40, 35, 30, 25, 20% w/w hexadecane. Even more in particular, the mixture of polypropylene glycol and hexadecane may comprise 70% w/w polypropylene glycol and about 30% w/w hexadecane.

In step (bi) according to any aspect of the present invention, the alcohol in the aqueous medium may contact the extracting medium for a time sufficient to extract the alcohol from the aqueous medium into the extracting medium. A skilled person may be capable of determining the amount of time needed for substantially all the alcohol to be extracted into the extracting medium. The time needed may be about, 1, 2, 3, 4, 5 6, 7, 8, 9 or 10 minutes. In some examples the time needed may be dependent on the amount of alcohol that may be extracted. In particular, the time needed to extract the alcohol from the aqueous medium into the extracting medium may be about 3 minutes.

The ratio of the extracting medium used to the amount of alcohol to be extracted may vary depending on how quick the extraction is to be carried out. In one example, the amount of extracting medium is equal to the amount of aqueous medium comprising the alcohol. After the step of contacting the extracting medium with the aqueous medium, the two phases (aqueous and organic) are separated using any means known in the art. In one example, the two phases may be separated using a separation funnel.

In one example, wherein the alcohol may be propanol, step (a) comprises
(ai) contacting the carbon source with a reaction mixture comprising
   a first acetogenic microorganism in an exponential growth phase;
   free oxygen; and
   a second acetogenic microorganism in a stationary phase
wherein the first and second acetogenic microorganism is capable of converting the carbon source to the acetate and/or ethanol;
(aii) contacting the acetate and/or ethanol from (ai) with a third microorganism capable of converting the acetate and/or ethanol to the propanol in the aqueous medium.

In another example, the alcohol may be at least one higher alcohol and step (a) comprises
(a1) contacting the carbon source with a reaction mixture comprising
   a first acetogenic microorganism in an exponential growth phase;
   free oxygen; and
   a second acetogenic microorganism in a stationary phase
wherein the first and second acetogenic microorganism is capable of converting the carbon source to the acetate and/or ethanol;
(a2) contacting the acetate and/or ethanol from step (a1) with a fourth microorganism capable of carrying out the ethanol carboxylate fermentation pathway and converting the acetate and/or ethanol from (a1) to form an acid;
(a3) contacting the acid from (a2) with the reaction mixture of step (a1)
wherein the first and/or second acetogenic microorganism is capable of converting the acid to the corresponding higher alcohol in the aqueous medium.

The third microorganism capable of converting acetate and/or ethanol to propanol and propionic acid may refer to any microorganism that may be able to carry out fermentative production of propanol. This third microorganism may be a propionogen. Propionogens are C3-producing microorganisms. In particular, propionogens refers to any microorganism which may be capable of converting syngas intermediates, such as ethanol and acetate, to propionic acid and propanol. The terms “propionogen” or “C3-producing microorganism” refers to microorganisms which, when contacted with a substrate, convert the substrate to propanol. These microorganisms may produce the appropriate enzymes intracellularly and/or extracellularly. These propanol producing microorganisms may be capable of utilising starting material for propanol fermentation that may be waste materials. For instance, ethanol and/or acetate derived from syngas may be utilized for the propanol production. This is particularly advantageous as inexpensive starting materials can be utilized that would originally have been considered waste. This also enables the removal of waste which consequently reduces environmental pollution. In one example, the propionogen according to any aspect of the present invention may use at least the methylmalonyl-succinate pathway or the lactate-acrylate pathway to produce propanol from acetate and/or ethanol.

In particular, the propionogen used according to any aspect of the present invention may be selected from the group consisting of Clostridium neopropionicum, Clostridium propionicum, Pelobacter propionicus, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii. More in particular, the propionogen may be Clostridium neopropionicum.

In one example, the third microorganism may be any eukaryotic or prokaryotic microorganism that may be genetically modified. More in particular, the third microorganism may be a strain selected from the group consisting of Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp., fungi and yeasts. Even more in particular, the third microorganism may be selected from Escherichia sp. For example, the third microorganism according to any aspect of the present invention may be Escherichia coli. The third microorganism may be a genetically modified organism comprising increased expression relative to the wild type cell of propionate CoA-transferase (AJ276553) (E₁), lactoyl-CoA dehydratase (JN244651-3) (E₂) and acryloyl-CoA reductase (JN244654-6) (E₃). Kandasamy V. (2013) discloses a method of producing a genetic organism as such. Kandasamy V. also discloses a means of measuring the expression of enzymes E₁, E₂ and E₃ to determine if any one of these enzymes have increased expression relative to the wild type cell.

In one example, the ethanol and/or acetate may be converted to the corresponding higher acid in the presence of the fourth microorganism capable of carrying out the ethanol-carboxylate fermentation pathway. The ethanol-carboxylate fermentation pathway is described in detail at least in Seedorf, H., et al., 2008. In particular, the fourth organism may be selected from the group consisting of Clostridium kluyveri, C. carboxidivorans and the like. These fourth microorganisms include microorganisms which in their wild-type form do not have an ethanol-carboxylate fermentation pathway, but have acquired this trait as a result of genetic modification. In particular, the fourth microorganism may be Clostridium kluyveri.

In another example, the fourth microorganism may be a wild type organism that expresses at least one enzyme selected from the group consisting of E₄ to E₁₄, wherein E₄ is an alcohol dehydrogenase (adh), E₅ is an acetaldehyde dehydrogenase (ald), E₆ is an acetoacetyl-CoA thiolase (thl), E₇ is a 3-hydroxybutyryl-CoA dehydrogenase (hbd), E₈ is a 3-hydroxybutyryl-CoA dehydratase (crt), E₉ is a butyryl-CoA dehydrogenase (bcd), E₁₀ is an electron transfer flavoprotein subunit (etf), E₁₁ is a coenzyme A transferase (cat), E₁₂ is an acetate kinase (ack), E₁₃ is phosphotransacetylase (pta) and E₁₄ is a transhydrogenase. In particular, the wild type fourth microorganism according to any aspect of the present invention may express at least E₅, E₆ and E₇. Even more in particular, the wild type fourth microorganism according to any aspect of the present invention may express at least E₇.

In another example, the fourth microorganism according to any aspect of the present invention may be a genetically modified organism that has increased expression relative to the wild type microorganism of at least one enzyme selected E₄ to E₁₄ wherein E₄ is an alcohol dehydrogenase (adh), E₅ is an acetaldehyde dehydrogenase (ald), E₆ is an acetoacetyl-CoA thiolase (thl), E₇ is a 3-hydroxybutyryl-CoA dehydrogenase (hbd), E₈ is a 3-hydroxybutyryl-CoA dehydratase (crt), E₉ is a butyryl-CoA dehydrogenase (bcd), E₁₀ is an electron transfer flavoprotein subunit (etf), is a coenzyme A transferase (cat), E₁₂ is an acetate kinase (ack) E₁₃ is phosphotransacetylase (pta) and E₁₄ is a transhydrogenase. In particular, the genetically modified fourth microorganism according to any aspect of the present invention may express at least enzymes E₅, E₆ and E₇. Even more in particular, the genetically modified fourth microorganism according to any aspect of the present invention may express at least E₇. The enzymes E₄ to E₁₄ may be isolated from Clostridium kluyveri. A skilled person may be capable of measuring the activity of each of these enzymes using methods known in the art. In particular, the activity of enzymes E₄ and E₅ may be measured using the assays taught at least in Hillmer P., 1972, Lurz R., 1979; the activity of enzyme E₅ may also be measured using the assay taught in Smith L. T., 1980; the activity of enzymes E₆ and E₇ may be measured using the assays taught at least in Sliwkowski M. X., 1984; the activity of E₇ may also be measured using the assay taught in Madan, V. K., 1972; the activity of E₈ may also be measured using the assay taught in Bartsch, R. G., 1961; the activity of enzymes E₉ and E₁₀ may be measured using the assay taught in Li, F., 2008; the activity of E₁₀ may also be measured using the assay taught in Chowdhury, 2013; the activity of may be measured using the assay taught in Stadman, 1953; the activity of E₁₂ may be measured using the assay taught in Winzer, K., 1997; the activity of E₁₃ may be measured using the assay taught in Smith L. T., 1976; and the activity of E₁₄ may be measured using the assay taught in Wang S, 2010.

The term “acetate” as used herein, refers to both acetic acid and salts thereof, which results inevitably, because as known in the art, since the microorganisms work in an aqueous environment, and there is always a balance between salt and acid present.

In particular, the second acetogenic microorganism in a post exponential phase may be in the stationary phase of the cell. The acetogenic cells in the log phase allow for any other acetogenic cells in the aqueous medium to produce acetate and/or ethanol in the presence of oxygen. The concentration of acetogenic cells in the log phase may be maintained in the reaction mixture. Therefore, at any point in time in the reaction, the reaction mixture comprises acetogenic cells in the log phase and acetogenic cells in another growth phase, for example in the stationary phase.

A skilled person would understand the different growth phases of microorganisms and the methods to measure them and identify them. In particular, most microorganisms in batch culture, may be found in at least four different growth phases; namely they are: lag phase (A), log phase or exponential phase (B), stationary phase (C), and death phase (D). The log phase may be further divided into the early log phase and mid to late log/exponential phase. The stationary phase may also be further distinguished into the early stationary phase and the stationary phase. For example, Cotter, J. L., 2009, Najafpour. G., 2006, Younesi, H., 2005, and Köpke, M., 2009 disclose different growth phases of acetogenic bacteria. In particular, the growth phase of cells may be measured using methods taught at least in Shuler ML, 1992 and Fuchs G., 2007.

The lag phase is the phase immediately after inoculation of the cells into a fresh medium, the population remains temporarily unchanged. Although there is no apparent cell division occurring, the cells may be growing in volume or mass, synthesizing enzymes, proteins, RNA, etc., and increasing in metabolic activity. The length of the lag phase may be dependent on a wide variety of factors including the size of the inoculum; time necessary to recover from physical damage or shock in the transfer; time required for synthesis of essential coenzymes or division factors; and time required for synthesis of new (inducible) enzymes that are necessary to metabolize the substrates present in the medium.

The exponential (log) phase of growth is a pattern of balanced growth wherein all the cells are dividing regularly by binary fission, and are growing by geometric progression. The cells divide at a constant rate depending upon the composition of the growth medium and the conditions of incubation. The rate of exponential growth of a bacterial culture is expressed as generation time, also the doubling time of the bacterial population. Generation time (G) is defined as the time (t) per generation (n=number of generations). Hence, G=t/n is the equation from which calculations of generation time derive. The exponential phase may be divided into the (i) early log phase and (ii) mid to late log/exponential phase. A skilled person may easily identify when a microorganism, particularly an acetogenic bacteria, enters the log phase. For example, the method of calculating the growth rate of acetogenic bacteria to determine if they are in the log phase may be done using the method taught at least in Henstra A. M., 2007. In particular, the microorganism in the exponential growth phase according to any aspect of the present invention may include cells in the early log phase and mid to late log/exponential phase.

The stationary phase is the phase where exponential growth ends as exponential growth cannot be continued forever in a batch culture (e.g. a closed system such as a test tube or flask). Population growth is limited by one of three factors: 1. exhaustion of available nutrients; 2. accumulation of inhibitory metabolites or end products; 3. exhaustion of space, in this case called a lack of “biological space”. During the stationary phase, if viable cells are being counted, it cannot be determined whether some cells are dying and an equal number of cells are dividing, or the population of cells has simply stopped growing and dividing. The stationary phase, like the lag phase, is not necessarily a period of quiescence. Bacteria that produce secondary metabolites, such as antibiotics, do so during the stationary phase of the growth cycle (Secondary metabolites are defined as metabolites produced after the active stage of growth).

The death phase follows the stationary phase. During the death phase, the number of viable cells decreases geometrically (exponentially), essentially the reverse of growth during the log phase.

In one example, where O₂ is present in the reaction mixture according to any aspect of the present invention, the first acetogenic bacteria may be in an exponential growth phase and the other acetogenic bacteria may be in any other growth phase in the lifecycle of an acetogenic microorganism. In particular, according to any aspect of the present invention, the acetogenic bacteria in the reaction mixture may comprise one acetogenic bacteria in an exponential growth phase and another in the stationary phase. In the presence of oxygen, without the presence of the acetogenic bacteria in an exponential growth, the acetogenic bacteria in the stationary phase may not be capable of producing acetate and/or ethanol. This phenomenon is confirmed at least by Brioukhanov, 2006, Imlay, 2006, Lan, 2013 and the like. The inventors thus surprisingly found that in the presence of acetogenic bacteria in an exponential growth, the acetogenic bacteria in any growth phase may aerobically respire and produce acetate and/or ethanol at more than or equal to the amounts produced when the reaction mixture was absent of oxygen. In one example, the acetogenic bacteria in the exponential growth phase may be capable of removing the free oxygen from the reaction mixture, providing a suitable environment (with no free oxygen) for the acetogenic bacteria in any growth phase to metabolise the carbon substrate to produce acetate and/or ethanol.

In another example, the aqueous medium may already comprise acetogenic bacteria in any growth phase, particularly in the stationary phase, in the presence of a carbon source. In this example, there may be oxygen present in the carbon source supplied to the aqueous medium or in the aqueous medium itself. In the presence of oxygen, the acetogenic bacteria may be inactive and not produce acetate and/or ethanol prior to the addition of the acetogenic bacteria in the exponential growth phase. In this very example, the acetogenic bacteria in the exponential growth phase may be added to the aqueous medium. The inactive acetogenic bacteria already found in the aqueous medium may then be activated and may start producing acetate and/or ethanol.

In a further example, the acetogenic bacteria in any growth phase may be first mixed with the acetogenic bacteria in the exponential growth phase and then the carbon source and/or oxygen added.

According to any aspect of the present invention, a microorganism in the exponential growth phase grown in the presence of oxygen may result in the microorganism gaining an adaptation to grow and metabolise in the presence of oxygen. In particular, the microorganism may be capable of removing the oxygen from the environment surrounding the microorganism. This newly acquired adaptation allows for the acetogenic bacteria in the exponential growth phase to rid the environment of oxygen and therefore produce acetate and ethanol from the carbon source. In particular, the acetogenic bacteria with the newly acquired adaptation allows for the bacteria to convert the carbon source to acetate and/or ethanol.

In one example, the acetogenic bacteria in the reaction mixture according to any aspect of the present impression may comprise a combination of cells: cells in the log phase and cells in the stationary phase. In the method according to any aspect of the present invention the acetogenic cells in the log phase may comprise a growing rate selected from the group consisting of 0.01 to 2 h⁻¹, 0.01 to 1 h⁻¹, 0.05 to 1 h⁻¹, 0.05 to 2 h⁻¹ 0.05 to 0.5 Wand the like. In one example, the OD₆₀₀ of the cells of the log phase acetogenic cells in the reaction mixture may be selected from the range consisting of 0.001 to 2, 0.01 to 2, 0.1 to 1, 0.1 to 0.5 and the like. A skilled person would be able to use any method known in the art to measure the OD₆₀₀ and determine the growth rate of the cells in the reaction mixture and/or to be added in the reaction mixture. For example, Koch (1994) may be used. In particular, bacterial growth can be determined and monitored using different methods. One of the most common is a turbidity measurement, which relies upon the optical density (OD) of bacteria in suspension and uses a spectrophotometer. The OD may be measured at 600 nm using a UV spectrometer.

In order to maintain the concentration of the first and second acetogenic bacteria in the reaction mixture, a skilled person may be capable of extracting a sample at fixed time points to measure the OD₆₀₀, pH, concentration of oxygen and concentration of ethanol and/or higher alcohols formed. The skilled person would then be able to add the necessary component(s) to maintain the concentration of first and second acetogenic bacteria in the reaction mixture and to ensure an optimum environment is maintained for the production of ethanol and/or acetate.

The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ERI2, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989. The first and second acetogenic bacteria used according to any aspect of the present invention may be the same or different bacteria. For example, in one reaction mixture the first acetogenic bacteria may be Clostridium ljungdahlii in the log phase and the second acetogenic bacteria may be Clostridium ljungdahlii in the stationary phase. In another example, in the reaction mixture the first acetogenic bacteria may be Clostridium ljungdahlii in the log phase and the second acetogenic bacteria may be Clostridium carboxidivorans in the stationary phase.

In the reaction mixture according to any aspect of the present invention, there may be oxygen present (i.e. aerobic conditions are used). It is advantageous to incorporate O₂ in the reaction mixture and/or gas flow being supplied to the reaction mixture as most waste gases including synthesis gas comprises oxygen in small or large amounts. It is difficult and costly to remove this oxygen prior to using synthesis gas as a carbon source for production of higher alcohols. The method according to any aspect of the present invention allows the production of at least one higher alcohol without the need to first remove any trace of oxygen from the carbon source. This allows for time and money to be saved.

More in particular, the O₂ concentration in the gas flow may be may be present at less than 1% by volume of the total amount of gas in the gas flow. In particular, the oxygen may be present at a concentration range of 0.000005 to 2% by volume, at a range of 0.00005 to 2% by volume, 0.0005 to 2% by volume, 0.005 to 2% by volume, 0.05 to 2% by volume, 0.00005 to 1.5% by volume, 0.0005 to 1.5% by volume, 0.005 to 1.5% by volume, 0.05 to 1.5% by volume, 0.5 to 1.5% by volume, 0.00005 to 1% by volume, 0.0005 to 1% by volume, 0.005 to 1% by volume, 0.05 to 1% by volume, 0.5 to 1% by volume, 0.55 to 1% by volume, 0.60 to 1% by volume, particularly at a range of 0.60 to 1.5%, 0.65 to 1%, and 0.70 to 1% by volume in the gas phase of the gas flow and/or in the medium. In particular, the acetogenic microorganism is particularly suitable when the proportion of O₂ in the gas phase/flow is about 0.00005, 0.0005, 0.005, 0.05, 0.5, 0.6, 0.7, 0.8, 0.9, 1, 1.5, 2% by volume in relation to the volume of the gas in the gas flow. A skilled person would be able to use any one of the methods known in the art to measure the volume concentration of oxygen in the gas flow. In particular, the volume of oxygen may be measured using any method known in the art. In one example, a gas phase concentration of oxygen may be measured by a trace oxygen dipping probe from PreSens Precision Sensing GmbH. Oxygen concentration may be measured by fluorescence quenching, where the degree of quenching correlates to the partial pressure of oxygen in the gas phase. Even more in particular, the first and second microorganisms according to any aspect of the present invention are capable of working optimally in the aqueous medium when the oxygen is supplied by a gas flow with concentration of oxygen of less than 1% by volume of the total gas, in about 0.015% by volume of the total volume of gas in the gas flow supplied to the reaction mixture.

According to any aspect of the present invention, the aerobic conditions in which the carbon source is converted to ethanol and/or acetate in the reaction mixture refers to gas surrounding the reaction mixture. The gas may comprise at least 1% by volume of the total gas of oxygen and other gases including carbon sources such as CO, CO₂ and the like.

The aqueous medium according to any aspect of the present invention may comprise oxygen. The oxygen may be dissolved in the medium by any means known in the art. In particular, the oxygen may be present at 0.5 mg/L in the absence of cells. In particular, the dissolved concentration of free oxygen in the aqueous medium may at least be 0.01 mg/L. In another example, the dissolved oxygen may be about 0.01, 0.02, 0.03, 0.04, 0.05, 0.1, 0.2, 0.3, 0.4, 0.5 mg/L. In particular, the dissolved oxygen concentration may be 0.01-0.5 mg/L, 0.01-0.4 mg/L, 0.01-0.3 mg/L, 0.01-0.1 mg/L. In particular, the oxygen may be provided to the aqueous medium in a continuous gas flow. More in particular, the aqueous medium may comprise oxygen and a carbon source comprising CO and/or CO₂. More in particular, the oxygen and a carbon source comprising CO and/or CO₂ is provided to the aqueous medium in a continuous gas flow. Even more in particular, the continuous gas flow comprises synthesis gas and oxygen. In one example, both gases are part of the same flow/stream. In another example, each gas is a separate flow/stream provided to the aqueous medium. These gases may be divided for example using separate nozzles that open up into the aqueous medium, frits, membranes within the pipe supplying the gas into the aqueous medium and the like. The oxygen may be free oxygen. According to any aspect of the present invention, ‘a reaction mixture comprising free oxygen’ refers to the reaction mixture comprising elemental oxygen in the form of O₂. The O₂ may be dissolved oxygen in the reaction mixture. In particular, the dissolved oxygen may be in the concentration of 5 ppm (0.000005% vol; 5×10⁻⁶). A skilled person may be capable of using any method known in the art to measure the concentration of dissolved oxygen. In one example, the dissolved oxygen may be measured by Oxygen Dipping Probes (Type PSt6 from PreSens Precision Sensing GmbH, Regensburg, Germany.

Step (aii) of the method according to any aspect of the present invention involves contacting the acetate and/or ethanol from step (ai) with a third microorganism capable of converting the acetate and/or ethanol to propanol. In particular, the third microorganism may be genetically modified to comprise increased expression relative to the wild type cell of enzymes necessary to carry out the methylmalonyl-succinate pathway or the lactate-acrylate pathway to produce propanol from acetate and/or ethanol.

According to any aspect of the present invention, the first, second, third and/or fourth microorganism may be a genetically modified microorganism. The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce propanol and/or propionic acid. The wild type microorganism relative to the genetically modified microorganism according to any aspect of the present invention may have none or no detectable activity of the enzymes that enable the genetically modified microorganism to produce propanol and/or propionic acid. As used herein, the term ‘genetically modified microorganism’ may be used interchangeably with the term ‘genetically modified cell’. The genetic modification according to any aspect of the present invention may be carried out on the cell of the microorganism.

The phrase “wild type” as used herein in conjunction with a cell or microorganism may denote a cell with a genome make-up that is in a form as seen naturally in the wild. The term may be applicable for both the whole cell and for individual genes. The term ‘wild type’ may thus also include cells which have been genetically modified in other aspects (i.e. with regard to one or more genes) but not in relation to the genes of interest. The term “wild type” therefore does not include such cells where the gene sequences of the specific genes of interest have been altered at least partially by man using recombinant methods. A wild type cell according to any aspect of the present invention thus refers to a cell that has no genetic mutation with respect to the whole genome and/or a particular gene. Therefore, in one example, a wild type cell with respect to enzyme E₁ may refer to a cell that has the natural/non-altered expression of the enzyme E₁ in the cell. The wild type cell with respect to enzyme E₂, E₃, E₄, E₅, E₆, E₇, E₈, E₉, E₁₀, E₁₁, E₁₂, E₁₃, E₁₄, etc. may be interpreted the same way and may refer to a cell that has the natural/non-altered expression of the enzyme E₂, E₃, E₄, E₅, E₆, E₇, E₈, E₉, E₁₀, E₁₁, E₁₂, E₁₃, E₁₄, etc. respectively in the cell.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more propanol and/or propionic acid than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (alcohol with at least 3 carbons) in the nutrient medium.

The term “second microorganism” or “third microorganism” or “fourth microorganism”, refers to a microorganism that is different from “the first microorganism” according to any aspect of the present invention. The numbers do not refer to the number of organisms that may be involved in the method according to any aspect of the present invention. For example, in a method of producing a higher alcohol according to any aspect of the present invention, a “first microorganism”, a “second microorganism” and a “fourth microorganism” are involved. There may not be a “third microorganism” involved in the method of producing higher alcohols according to any aspect of the present invention. The use of the numbers to refer to the microorganisms may thus be taken to differentiate between the different microorganisms involved according to any aspect of the present invention but not to the number of organisms involved. For example, the use of “fourth microorganism” in the method according to any aspect of the present invention does not mean there are four organisms used in the method but that the specific fourth microorganism may be present in the method.

In one example, where propanol is the alcohol produced according to any aspect of the present invention, the first, second and third microorganism may be present in one fermenter in the presence of oxygen. The product, aqueous medium comprising the propanol may then be transferred to another vessel for carrying out (b) extraction using the extracting medium.

In a further example, where propanol is the alcohol produced according to any aspect of the present invention, the first, second and third microorganism may be present in one fermenter in the presence of oxygen. The product, propanol in the aqueous medium may then be directly extracted from the medium containing the cells. The cells may then be recycled for production of the propanol.

In one example, where propanol is the alcohol produced according to any aspect of the present invention, the first and second microorganism may be present in a first fermenter and the third microorganism in a second fermenter. In fermenter 1, the first and second microorganisms come in contact with the carbon source to produce acetate and/or ethanol. Ethanol and/or acetate may then be brought into contact with a third microorganism in fermenter 2 to produce propanol. A cycle may be created wherein the acetate and/or ethanol produced in fermenter 1 may be regularly fed into fermenter 2, the acetate and/or ethanol in fermenter 2 may be converted to propanol. Oxygen may be added into fermenter 2 to enable the third microorganism to convert acetate to propanol.

Similarly, in fermenter 1 the first and second microorganism may come in contact with the carbon source comprising CO to produce acetate and/or ethanol. Ethanol and/or acetate may then be brought into contact with a third microorganism in fermenter 2 to produce propanol. A cycle may be created wherein the acetate and/or ethanol produced in fermenter 1 may be regularly fed into fermenter 2, the acetate and/or ethanol in fermenter 2 may be converted to propanol. CO fed into fermenter 1 may be transferred into fermenter 2 together with the acetate and/or ethanol. No special extraction method may be needed between the two fermenters.

In one example, where a higher alcohol is produced according to any aspect of the present invention, the first, second and fourth microorganism may be present in a first fermenter. In the fermenter the first and second microorganisms come in contact with the carbon source first to produce acetate and/or ethanol. Ethanol and/or acetate then contacts the fourth microorganism in the same fermenter to produce at least one acid. The acid then contacts then produces at least one corresponding higher alcohol when it contacts the first and second microorganisms again. No special extraction method may be needed as the fourth microorganism has surprisingly been found to convert acetate and/or ethanol to at least one acid in the presence of CO.

In a further example, where a higher alcohol is produced according to any aspect of the present invention, the first and second microorganism may be present in a first fermenter and the fourth microorganism in a second fermenter. In fermenter 1, the first and second microorganisms come in contact with the carbon source to produce acetate and/or ethanol. Ethanol and/or acetate may then be brought into contact with a fourth microorganism in fermenter 2 to produce at least one acid. The acid may then be fed back into fermenter 1 to produce at least one higher alcohol. A cycle may be created wherein the acetate and/or ethanol produced in fermenter 1 may be regularly fed into fermenter 2, the acetate and/or ethanol in fermenter 2 may be converted to at least one acid and the acid in fermenter 2 fed back into fermenter 1. Similarly, in fermenter 1 the first and second microorganism may come in contact with the carbon source comprising CO to produce acetate and/or ethanol. Ethanol and/or acetate may then be brought into contact with a fourth microorganism in fermenter 2 to produce at least one acid. The acid may then be optionally extracted and fed back into fermenter 1 to convert the acid to the desired higher alcohol. A cycle may be created wherein the acetate and/or ethanol produced in fermenter 1 may be regularly fed into fermenter 2, the acetate and/or ethanol in fermenter 2 may be converted to at least one acid and the acid in fermenter 2 fed back into fermenter 1. CO fed into fermenter 1 may be transferred into fermenter 2 together with the acetate and/or ethanol. No special extraction method may be needed as the fourth microorganism has surprisingly been found to convert acetate and/or ethanol to at least one acid in the presence of CO.

In yet another example, where a higher alcohol is produced according to any aspect of the present invention, the media is being recycled between fermenters 1 and 2. Therefore, the ethanol and/or acetate produced in fermenter 1 may be fed into fermenter 2 and the acid produced in fermenter 2 may be fed back into fermenter 1. In the process of recycling the media, CO from fermenter 1 may be introduced into fermenter 2. Also, the acids produced in fermenter 2 may be consequently reintroduced into fermenter 1. The fourth microorganisms in fermenter 2 may be able to continue producing acids from acetate and ethanol in the presence of the CO recycled from fermenter 1 into fermenter 2. The accumulated higher alcohols in fermenters 1 and 2 may then be extracted by means known in the art.

In a further example, there may be three containers present to carry out the method according to any aspect of the present invention. The first and second microorganism may be present in a first fermenter, the fourth microorganism in a second fermenter and a third fermenter with the first and second microorganisms. In fermenter 1, the first and second microorganisms come in contact with the carbon source to produce acetate and/or ethanol. Ethanol and/or acetate may then be brought into contact with a fourth microorganism in fermenter 2 to produce at least one acid. The acid may then be fed into fermenter 3 to produce at least one higher alcohol.

In the production of the acid and/or higher alcohol from the carbon source a combination of bacteria may be used. There may be more than one acetogenic bacteria present in combination with one or more fourth microorganisms. In another example, there may be more than one type of acetogenic bacteria present and only one type of fourth microorganism. In yet another example, there may be more than one fourth microorganism present in combination with only one acetogenic bacteria.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. Similarly, a decreased activity of an enzyme refers to decreased intracellular activity. In one example, the increased expression of an enzyme according to any aspect of the present invention may be 5, 10, 15, 20, 25, 30, 25, 40, 45, 50, 55, 60, 65, 70, 75, 80, 85, 90, 95 or 100% more relative to the expression of the enzyme in the wild type cell. Similarly, the decreased expression of an enzyme according to any aspect of the present invention may be 5, 10, 15, 20, 25, 30, 25, 40, 45, 50, 55, 60, 65, 70, 75, 80, 85, 90, 95 or 100% less relative to the expression of the enzyme in the wild type cell.

The culture medium to be used must be suitable for the requirements of the particular strains. Descriptions of culture media for various microorganisms are given in “Manual of Methods for General Bacteriology”.

All percentages (%) are, unless otherwise specified, mass percent.

With respect to the source of substrates comprising carbon dioxide and/or carbon monoxide, a skilled person would understand that many possible sources for the provision of CO and/or CO₂ as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight—% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount.

Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

A skilled person would understand the other conditions necessary to carry out the method according to any aspect of the present invention. In particular, the conditions in the container (e.g. fermenter) may be varied depending on the first, second and third microorganisms used. The varying of the conditions to be suitable for the optimal functioning of the microorganisms is within the knowledge of a skilled person.

In one example, the method according to any aspect of the present invention may be carried out in an aqueous medium with a pH between 5 and 8, 5.5 and 7. The pressure may be between 1 and 10 bar. The term “contacting”, as used herein, means bringing about direct contact between the cell according to any aspect of the present invention and the medium comprising the carbon source in step (a) and/or the direct contact between the third microorganism and the acetate and/or ethanol from step (a) in step (b). For example, the cell, and the medium comprising the carbon source may be in different compartments in step (a). In particular, the carbon source may be in a gaseous state and added to the medium comprising the cells according to any aspect of the present invention.

In particular, the aqueous medium may comprise the cells and a carbon source comprising CO and/or CO₂ for step (a) to be carried out. More in particular, the carbon source comprising CO and/or CO₂ is provided to the aqueous medium comprising the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

The overall efficiency, alcohol productivity and/or overall carbon capture of the method of the present invention may be dependent on the stoichiometry of the CO₂, CO, and H₂ in the continuous gas flow.

The continuous gas flows applied may be of composition CO₂ and H₂. In particular, in the continuous gas flow, concentration range of CO₂ may be about 10-50%, in particular 3% by weight and H₂ would be within 44% to 84%, in particular, 64 to 66.04% by weight. In another example, the continuous gas flow can also comprise inert gases like N₂, up to a N₂ concentration of 50% by weight.

The term ‘about’ as used herein refers to a variation within 20 percent. In particular, the term “about” as used herein refers to +/−20%, more in particular, +/−10%, even more in particular, +/−5% of a given measurement or value.

A skilled person would understand that it may be necessary to monitor the composition and flow rates of the streams at relevant intervals. Control of the composition of the stream can be achieved by varying the proportions of the constituent streams to achieve a target or desirable composition. The composition and flow rate of the blended stream can be monitored by any means known in the art. In one example, the system is adapted to continuously monitor the flow rates and compositions of at least two streams and combine them to produce a single blended substrate stream in a continuous gas flow of optimal composition, and means for passing the optimised substrate stream to the fermenter.

Microorganisms which convert CO₂ and/or CO to acetate and/or ethanol, in particular acetate, as well as appropriate procedures and process conditions for carrying out this metabolic reaction is well known in the art. Such processes are, for example described in WO9800558, WO2000014052 and WO2010115054.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell.

In particular, the reaction mixture according to any aspect of the present invention (i.e. mixture of the first microorganism—the acetogenic organism in log phase, the second microorganism—the acetogenic organism in stationary phase, the carbon source in the presence of oxygen can be employed in any known bioreactor or fermenter to carry out any aspect of the present invention. The reaction mixture may further comprise a fourth microorganism to result in higher alcohols being produced in the fermenter. In another example, the reaction mixture may further comprise a third microorganism to result in propanol being produced in the fermenter

‘Higher alcohols’ as used herein refers to alcohols that contain 4 to 10 carbon atoms and may be somewhat viscous, or oily, and have heavier fruity odours. Higher alcohols may include but are not limited to butanol, pentanol, hexanol, heptanol, octanol, nonanol, decanol and the like. More in particular, the higher alcohol may be selected from the group consisting of 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, 6-methyl-1-heptanol and combinations thereof.

According to any aspect of the present invention, the ‘corresponding higher alcohol’ refers to an alcohol with the same number of carbon atoms as that of the acid from which the corresponding higher alcohol is formed. For example, butanoic acid may be converted to the corresponding alcohol-butanol; hexanoic acid may be converted to the corresponding alcohol-hexanol; heptanoic acid may be converted to the corresponding alcohol-heptanol; octanoic acid may be converted to the corresponding alcohol-octanol; nonanoic acid may be converted to the corresponding alcohol-nonanol; decanoic acid may be converted to the corresponding alcohol-decanol and the like.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Co-cultivation of Clostridium ljungdahlii and Clostridium kluyveri in defined medium on hydrogen and carbon dioxide to produce butanol

C. ljungdahlii as first organism was autotrophically cultivated in defined medium in order to produce acetate and ethanol. After a given time, C. kluyveri as second organism was then inoculated in the same reactor for the conversion of acetate and ethanol to buyrate and hexanoate. In the following, C. ljungdahlii then converts butyrate to butanol.

A defined medium was used for the co-cultivation of both microorganisms consisting of 2 g/L (NH₄)₂HPO₄, 0.2 g/L NaCl, 0.15 g/l KCl, 1 g/l KOH, 0.5 g/L MgCl₂×6H₂O, 0.2 g/L CaCl₂)×2H₂O, 15 mg/L FeCl₂×4H₂O, 0.4 g/L L-cysteine-HCl, 0.4 g/L Na₂S×9H₂O, 3 mg/L boric acid, 2 mg/L CoCl₂×6H₂O, 1 mg/L ZnSO₄×7 H₂O, 0.3 mg/L Na₂MoO₄×2 H₂O, 0.3 mg/L MnSO₄×H₂O, 0.2 mg/L NiCl₂×6 H₂O, 0.1 mg/L CuCl₂×2 H₂O, 0.1 mg/L Na₂SeO₃, 106 μg/L biotin, 5 μg/L folic acid, 2.5 μg/L pyridoxine-HCl, 266 μg/L thiamine-HCl×H₂O, 12.5 μg/L riboflavin, 12.5 μg/L nicotinic acid, 413 μg/L Ca-pantothenoic acid, 12.5 μg/L vitamine B12, 12.5 μg/L p-aminobenzoic acid, 15 μg/L lipioic acid.

The autotrophic cultivation was performed in 250 mL defined medium in a 500 mL serum bottle that was continuously gassed with synthesis gas consisting of 67% H₂ and 33% CO₂ at a rate of 1 L/h. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The pH was held in a range of pH 5.0-6.5 by continuous addition of an anaerobic stock solution of KOH (40 g/L).

At the beginning of the experiment, C. ljungdahlii was inoculated with an OD₆₀₀ of 0.1 with autotrophically grown cells. Therefore, C. ljungdahlii was grown in complex medium under continuous gassing with synthesis gas consisting of 67% H₂ and 33% CO₂ at a rate of 3 L/h in 1 L serum bottles with 500 mL complex medium. A complex medium was used consisting of 1 g/L NH₄Cl, 0.1 g/L KCl, 0.2 g/L MgSO₄×7 H₂O, 0.8 g/L NaCl, 0.1 g/L KH₂PO₄, 20 mg/L CaCl₂)×2H₂O, 20 g/L MES, 1 g/L yeast extract, 0.4 g/L L-cysteine-HCl, 0.4 g/L Na₂S×9H₂O, 20 mg/L nitrilotriacetic acid, 10 mg/L MnSO₄×H₂O, 8 mg/L (NH₄)₂Fe(SO₄)₂×6 H₂O, 2 mg/L CoCl₂×6 H₂O, 2 mg/L ZnSO₄×7 H₂O, 0.2 mg/L CuCl₂×2 H₂O, 0.2 mg/L Na₂MoO₄×2 H₂O, 0.2 mg/L NiCl₂×6 H₂O, 0.2 mg/L Na₂SeO₄, 0.2 mg/L Na₂WO4×2 H₂O, 20 μg/L biotin, 20 μg/L folic acid, 100 μg/L pyridoxine-HCl, 50 μg/L thiamine-HCl×H₂O, 50 μg/L riboflavin, 50 μg/L nicotinic acid, 50 μg/L Ca-pantothenoic acid, 1 μg/L vitamine B12, 50 μg/L p-aminobenzoic acid, 50 μg/L lipoic acid. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The cells were harvested in the late-logarithmic phase with an OD₆₀₀ of 0.67 and a pH of 4.69 by anaerobic centrifugation (4500 min⁻¹, 4300 g, 20° C., 10 min). The supernatant was discarded and the pellet was resuspended in 10 mL of above described defined medium. This cell suspension was then used to inoculate the co-culture experiment.

Parallel to that, C. kluyveri were grown heterotrophically in 200 mL complex medium in 500 mL serum bottles on acetate and ethanol. A complex medium was used consisting of 0.25 g/L NH₄Cl, 0.2 g/L MgSO₄×7 H₂O, 0.31 g/L K₂HPO₄, 0.23 g/L KH₂PO₄, 2.5 g/L NaHCO₃, 1 g/L yeast extract, 10 g/L K-acetate, 20 g/l ethanol, 0.25 g/L L-cysteine-HCl, 1.5 mg/L FeCl₂×4 H₂O, 70 μg/L ZnCl₂×7 H₂O, 100 μg/L MnCl₂×4 H₂O, 6 μg/L boric acid, 190 μg/L CoCl₂×6 H₂O, 2 μg/L CuCl₂×6 H₂O, 24 μg/L NiCl₂×6 H₂O, 36 μg/L Na₂MoO₄×2 H₂O, 3 μg/L Na₂SeOO₃×5 H₂O, 4 μg/L Na₂WO4×2 H₂O, 100 μg/L vitamin B12, 80 μg/L p-aminobenzoic acid, 20 μg/L biotin, 200 μg/L nicotinic acid, 100 μg/L Ca-pantothenoic acid, 300 μg/L pyridoxine-HCl, 200 μg/L thiamine-HCl×H₂O. The serum bottle was continuously shaken in an open water bath Innova 3100 from New Brunswick Scientific at 37° C. and a shaking rate of 100 min⁻¹. The cells were harvested in the late-logarithmic phase with an OD₆₀₀ of 0.81 and a pH of 5.96 by anaerobic centrifugation (4500 min⁻¹, 4300 g, 20° C., 10 min). The supernatant was discarded and the pellet was resuspended in 10 mL of above described defined medium. This cell suspension was then used to inoculate the co-culture experiment with an OD₆₀₀ of 0.2 after 96 hours of the running experiment.

During the experiment samples of 5 mL were taken for the determination of OD₆₀₀, pH and product concentrations. The latter were determined by quantitative ¹H-NMR-spectroscopy.

After inoculation of C. ljungdahlii, cells began to grow and continuously produced acetate. Concomitant to the production of acetate, ethanol was produced in a lower rate compared to the production of acetate. After 96 hours C. kluyveri was then inoculated into the reactor a decrease of ethanol concentration was measured in the following experiment. The simultaneous production of butyrate (max. 1163 mg/L) and hexanoate (max. 136 mg/L) was then measured in the following 113 hours of the experiment. Parallel to the production of butyrate by C. kluyveri, C. ljungdahlii converted butyrate to butanol to a maximum concentration of 20 mg/L butanol at the end of the experiment.

TABLE 1
Results of Example 1

| | | | NMR-analytics | | | | | |
|---|---|---|---|---|---|---|---|---|
| Process | | | Acetate, | Ethanol, | Butyrate, | n-Butanol, | Hexanoate, | Hexanol, |
| time, h | pH | OD600 | mg/L | mg/L | mg/L | mg/L | mg/L | mg/L |
| 0.0 | 6.37 | 0.11 | 4 | 2 | n.d. | n.d. | n.d. | n.d. |
| 19.5 | 5.49 | 0.12 | 818 | 10 | n.d. | n.d. | n.d. | n.d. |
| 40.3 | 5.49 | 0.21 | 1930 | 51 | n.d. | n.d. | n.d. | n.d. |
| 63.8 | 5.10 | 0.43 | 5005 | 160 | n.d. | n.d. | n.d. | n.d. |
| 79.5 | 5.85 | 0.49 | 8444 | 260 | n.d. | n.d. | n.d. | n.d. |
| 95.0 | 5.95 | 0.58 | 8984 | 291 | n.d. | n.d. | n.d. | n.d. |
| 96.0 | 5.90 | 0.78 | 9299 | 316 | 16 | n.d. | 23 | n.d. |
| 121.5 | 6.28 | 0.71 | 11030 | 9 | 801 | 10 | 81 | n.d. |
| 142.3 | 6.29 | 0.71 | 12238 | 8 | 934 | 14 | 87 | n.d. |
| 160.3 | 6.30 | 0.67 | 13096 | 9 | 1006 | 16 | 103 | n.d. |
| 185.5 | 6.28 | 0.57 | 16860 | 13 | 1143 | 17 | 134 | n.d. |
| 209.0 | 6.23 | 0.52 | 19275 | 17 | 1163 | 16 | 136 | n.d. |
| 234.0 | 5.82 | 0.42 | 18368 | 19 | 991 | 15 | 108 | n.d. |
| 259.3 | 5.72 | 0.34 | 18744 | 20 | 1005 | 15 | 116 | n.d. |

(n.d. = not detected)

### Example 2

Co-Cultivation of Clostridium ljungdahlii and Clostridium kluyveri in Complex Medium with CO-Containing Gas to Produce Butanol and Hexanol

C. ljungdahlii as first organism was autotrophically cultivated in complex medium in order to produce acetate and ethanol. After a given time, C. kluyveri as second organism was then inoculated in the same reactor for the conversion of acetate and ethanol to buyrate and hexanoate. In the following, C. ljungdahlii then converts butyrate to butanol and hexanoate to hexanol.

A complex medium was used for the co-cultivation of both microorganisms consisting of 1 g/L NH₄Cl, 0.1 g/L KCl, 0.2 g/L MgSO₄×7 H₂O, 0.8 g/L NaCl, 0.1 g/L KH₂PO₄, 20 mg/L CaCl₂×2 H₂O, 20 g/L MES, 1 g/L yeast extract, 0.4 g/L L-cysteine-HCl, 0.4 g/L Na₂S×9H₂O, 20 mg/L nitrilotriacetic acid, 10 mg/L MnSO₄×H₂O, 8 mg/L (NH₄)₂Fe(SO₄)₂×6 H₂O, 2 mg/L CoCl₂×6 H₂O, 2 mg/L ZnSO₄×7 H₂O, 0.2 mg/L CuCl₂×2 H₂O, 0.2 mg/L Na₂MoO₄×2 H₂O, 0.2 mg/L NiCl₂×6 H₂O, 0.2 mg/L Na₂SeO₄, 0.2 mg/L Na₂WO₄×2 H₂O, 20 μg/L biotin, 20 μg/L folic acid, 100 μg/L pyridoxine-HCl, 50 μg/L thiamine-HCl×H₂O, 50 μg/L riboflavin, 50 μg/L nicotinic acid, 50 μg/L Ca-pantothenoic acid, 1 μg/L vitamine B12, 50 μg/L p-aminobenzoic acid, 50 μg/L lipoic acid.

The autotrophic cultivation was performed in 500 mL complex medium in a 1 L serum bottle that was continuously gassed with synthesis gas consisting of 5% H₂, 25% CO₂, 25% CO and 45% N₂ at a rate of ˜12 L/h (≥0.5 ppm). The gas was introduced into the liquid phase by a microbubble disperser with a pore diameter of 10 μm. The serum bottle was continuously shaken in an open water bath Innova 3100 from New Brunswick Scientific at 37° C. and a shaking rate of 120 min⁻¹. The pH was not controlled during this experiment.

At the beginning of the experiment, C. ljungdahlii was inoculated with an OD₆₀₀ of 0.1 with autotrophically grown cells. Therefore, C. ljungdahlii was grown in above described complex medium under continuous gassing with synthesis gas consisting of 67% H₂ and 33% CO₂ at a rate of 3 L/h in 1 L serum bottles with 500 mL complex medium. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The cells were harvested in the late-logarithmic phase with an OD₆₀₀ of 0.51 and a pH of 5.04 by anaerobic centrifugation (4500 min⁻¹, 4300 g, 20° C., 10 min). The supernatant was discarded and the pellet was resuspended in 10 mL of above described complex medium. This cell suspension was then used to inoculate the co-culture experiment.

Parallel to that, C. kluyveri was grown heterotrophically in 200 mL complex medium in 500 mL serum bottles on acetate and ethanol. A complex medium was used consisting of 0.25 g/L NH₄Cl, 0.2 g/L MgSO₄×7 H₂O, 0.31 g/L K₂HPO₄, 0.23 g/L KH₂PO₄, 2.5 g/L NaHCO₃, 1 g/L yeast extract, 10 g/L K-acetate, 20 g/l ethanol, 0.25 g/L L-cysteine-HCl, 1.5 mg/L FeCl₂×4 H₂O, 70 μg/L ZnCl₂×7 H₂O, 100 μg/L MnCl₂×4 H₂O, 6 μg/L boric acid, 190 μg/L CoCl₂×6 H₂O, 2 μg/L CuCl₂×6 H₂O, 24 μg/L NiCl₂×6 H₂O, 36 μg/L Na₂MoO₄×2 H₂O, 3 μg/L NazSeOO₃×5 H₂O, 4 μg/L Na₂WO₄×2 H₂O, 100 μg/L vitamine B12, 80 μg/L p-aminobenzoic acid, 20 μg/L biotin, 200 μg/L nicotinic acid, 100 μg/L Ca-pantothenoic acid, 300 μg/L pyridoxine-HCl, 200 μg/L thiamine-HCl×H₂O. The serum bottle was continuously shaken in an open water bath Innova 3100 from New Brunswick Scientific at 37° C. and a shaking rate of 100 min⁻¹. The cells were harvested in the late-logarithmic phase with an OD₆₀₀ of 0.54 and a pH of 6.60 by anaerobic centrifugation (4500 min⁻¹, 4300 g, 20° C., 10 min). The supernatant was discarded and the pellet was resuspended in 10 mL of above described complex medium. This cell suspension was then used to inoculate the co-culture experiment after 240 hours of the running experiment.

During the experiment samples of 5 mL were taken for the determination of OD₆₀₀, pH and product concentrations. The latter were determined by quantitative ¹H-NMR-spectroscopy.

After inoculation of C. ljungdahlii, cells began to grow and continuously produced acetate to a concentration of ˜3 g/L and ethanol to a concentration of ˜0.5 g/L after 71 hours. In the following time course of the experiment, acetate was completely converted to ethanol up to a concentration of 4.8 g/L after 240 hours. At a process time of 240 hours, C. kluyveri was then inoculated into the reactor. As this organism needs acetate besides ethanol as substrate, simultaneous to the inoculation of C. kluyveri approximately 3 g/L acetate (in the form of Na-acetate) were brought into the reactor anaerobically. In the following time course of the experiment, the production of butyrate and hexanoate up to concentrations of 1.6 g/L each were measured. Parallel to the production of butyrate and hexanoate by C. kluyveri, C. ljungdahlii converted butyrate to butanol to a maximum concentration of 690 mg/L butanol and converted hexanaote to hexanol to a maximum concentration of 1478 mg/L hexanol.

TABLE 2
Results of Example 2

| | | | NMR-analytics | | | | | |
|---|---|---|---|---|---|---|---|---|
| Process | | | Acetate, | Ethanol, | Butyrate, | n-Butanol, | Hexanoate, | Hexanol, |
| time, h | pH | OD600 | mg/L | mg/L | mg/L | mg/L | mg/L | mg/L |
| 0.0 | 6.13 | 0.11 | 26 | 3 | n.d. | n.d. | n.d. | n.d. |
| 18.0 | 5.89 | 0.55 | 1063 | 18 | n.d. | n.d. | n.d. | n.d. |
| 42.0 | 5.58 | 1.02 | 2353 | 79 | n.d. | n.d. | n.d. | n.d. |
| 71.3 | 5.31 | 1.34 | 3081 | 534 | n.d. | n.d. | n.d. | n.d. |
| 117.5 | 5.39 | 1.78 | 2612 | 1946 | n.d. | n.d. | n.d. | n.d. |
| 162.0 | 5.87 | 1.88 | 665 | 4153 | n.d. | n.d. | n.d. | n.d. |
| 192.0 | 6.02 | 1.85 | 43 | 4747 | n.d. | n.d. | n.d. | n.d. |
| 240.0 | 6.03 | 1.19 | 28 | 4805 | n.d. | n.d. | n.d. | n.d. |
| 240.0 | 6.03 | 1.17 | 3209 | 4775 | 134 | n.d. | 46 | n.d. |
| 258.0 | 6.24 | 1.22 | 1078 | 1727 | 46 | 522 | 1380 | 457 |
| 283.5 | 6.49 | 1.24 | 331 | 112 | 1380 | 690 | 1590 | 1478 |
| 330.0 | 6.50 | 0.80 | 343 | 110 | 1590 | 603 | 1344 | 1165 |

(n.d. = not detected)

### Example 3

Growth and Production of Acetate and Ethanol by Clostridium autoethanogenum on Synthesis Gas with Oxygen

For the biotransformation of hydrogen and carbon dioxide to acetic acid and ethanol the homoacetogenic bacterium Clostridium autoethanogenum was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock of C. autoethanogenum. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 72 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was resuspended in fresh medium. For the main culture, as many cells from the preculture as necessary for an OD₆₀₀ₙₘ of 0.1 were transferred in 500 mL medium with additional 400 mg/L L-cysteine-hydrochlorid. The chemolithoautotrophic cultivation was carried out in a 1 L pressure-resistant glass bottle at 37° C., 150 rpm and a ventilation rate of 1 L/h with a premixed gas with 66.95% H₂, 33% CO₂, 0.05% O₂ in an open water bath shaker for 41 h. The gas was discharged into the medium through a sparger with a pore size of 10 μm, which was mounted in the center of the reactors. Culturing was carried out with no pH control. During cultivation several 5 mL samples were taken to determinate OD₆₀₀ₙₘ, pH and product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed by an increase of the OD₆₀₀ₙₘ from 0.08 to 0.76 in 41 h, which correlates with a growth rate of p=0.054 h⁻¹. The concentration of acetate increased from 37 mg/L to 6600 mg/L and the concentration of ethanol increased from 4 mg/L to 120 mg/L. Over the whole cultivation period the dissolved oxygen concentration was 0.00 mg/L.

In a similar technical setting with the same parameters (medium composition, volume, bottle, gas, ventilation rate, temperature, shaking frequency), but without cells in the medium, a dissolved oxygen concentration of 0.01 mg/L was measured.

### Example 4

Growth and Acetate Production by Clostridium ljungdahffi on Synthesis Gas with 0.15% Oxygen

For the biotransformation of hydrogen and carbon dioxide to acetic acid the homoacetogenic bacterium Clostridium ljungdahlii was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock of C. ljungdahlii. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 72 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was washed with 10 ml medium and centrifuged again. For the main culture, as many washed cells from the preculture as necessary for an OD₆₀₀ₙₘ of 0.1 were transferred in 200 mL medium with additional 400 mg/L L-cysteine-hydrochlorid. The chemolithoautotrophic cultivation was carried out in a 250 mL pressure-resistant glass bottles at 37° C., 150 rpm and a ventilation rate of 1 L/h with a premixed gas with 66.85% H₂, 33% CO₂, 0.15% O₂ in an open water bath shaker for 47 h. The gas was discharged into the medium through a sparger with a pore size of 10 μm, which was mounted in the center of the reactors. Culturing was carried out with no pH control. During cultivation several 5 mL samples were taken to determinate OD₆₀₀ₙₘ, pH and product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed by an increase of the OD₆₀₀ₙₘ from 0.10 to 0.45, which correlates with a growth rate of p=0.032 h⁻¹. The concentration of acetate increased from 7 mg/L to 2347 mg/L and the concentration of ethanol increased from 2 mg/L to 319 mg/L. Over the whole cultivation period the dissolved oxygen concentration was 0.00 mg/L.

In a similar technical setting with the same parameters (medium composition, volume, bottle, gas, ventilation rate, temperature, shaking frequency), but without cells in the medium, a dissolved oxygen concentration of 0.03 mg/L was measured.

### Example 5

Production of propionic acid and propanol on synthesis gas with oxygen with Clostridium autoethanogenum and Clostridium neopropionicum

For the biotransformation of hydrogen and carbon dioxide to propionic acid and propanol the homoacetogenic bacterium Clostridium autoethanogenum was cultivated on synthesis gas in combination with Clostridium neopropionicum in a co-cultivation phase. All cultivation steps were carried out under anaerobic or microaerophile conditions in pressure-resistant glass bottles that could be closed airtight with a butyl rubber stopper.

For the cultivation of C. autoethanogenum 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂)×2H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochloride and 400 mg/L Na₂S×9H₂O was inoculated with 5 mL of a frozen cryo stock. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 64 h till OD₆₀₀ₙₘ of 0.36. The gas was discharged into the medium through a sparger with a pore size of 10 μm, which was mounted in the center of the reactors. Then the cell suspension was centrifuged, washed with fresh ATCC1754-medium and centrifuged again.

For the main culture of C. autoethanogenum 500 ml ATCC1754-medium with additional 400 mg/L L-cysteine-hydrochloride and 400 mg/L Na₂S×9H₂O was inoculated with washed cells from the first preculture to an OD₆₀₀ₙₘ of 0.1. The chemolithoautotrophic cultivation was carried out in a 1 L pressure-resistant glass bottle at 37° C., 100 rpm and a ventilation rate of 1 L/h with a premixed gas with 66.85% H₂, 33% CO₂, 0.15% O₂ in an open water bath shaker for 51 h till OD₆₀₀ₙₘ of 0.19 and pH 5.7. The gas was discharged into the medium through a sparger with a pore size of 10 μm, which was mounted in the center of the reactors.

For the first preculture of C. neopropionicum 2×100 ml DSMZ318 medium (pH 7.4; 0.61 g/l NaCl, 0.047 g/l MgCl₂, 0.30 g/l KH₂PO₄, 1.00 g/l NH₄Cl, 0.081 g/l CaCl₂)×2H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl₃×6 H₂O, 1 mg/L MnCl₂×4 H₂O, 0.24 mg/L CoCl₂×6 H₂O, 1 mg/L ZnCl₂, 0.25 mg/L CuCl₂×2 H₂O, 0.1 mg/L H₃B03, 0.24 mg/L Na₂MoO₄×2 H₂O, 1.2 mg/L NiCl₂×6 H₂O, 0.26 mg/L Na₂SeO₃×5 H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B12, 0.05 mg/L p-aminobenzoate, 0.05 mg/L lipoic acid, 0.25 g/L cysteine-HCl×H₂O) in a 250 ml bottle was inoculated with 5 ml of a frozen cryoculture and flushed with a premixed gas with 67% H₂, 33% CO₂ to an overpressure of 0.8 bar. The culture was incubated at 30° C. and 100 rpm in an open water bath shaker for 19 h till an OD₆₀₀ₙₘ>0.14.

For a second preculture of Clostridium neopropionicum 5×200 ml of fresh DSMZ318 medium in a 500 ml bottle was inoculated with centrifuged cells from the first preculture to an OD₆₀₀ₙₘ of 0.02 and flushed with a premixed gas with 67% H₂, 33% CO₂ to an overpressure of 0.8 bar. This growing culture was incubated at 30° C. and 100 rpm in an open water bath shaker for 24 h till an OD₆₀₀ₙₘ>0.26. Then the cell suspension was centrifuged, washed with fresh ATCC1754-medium and centrifuged again.

For the co-cultivation culture, as many washed cells from the second preculture of C. neopropionicum as necessary for an OD₆₀₀ₙₘ of 0.2 were added to the continuously aerated main culture of C. autoethanogenum after 51 h of cultivation. The cultivation was carried out in a 1 L pressure-resistant glass bottle at 37° C., 100 rpm and a ventilation rate of 1 L/h with a premixed gas with 66.85% H₃, 33% CO₂, 0.15% O₂ in an open water bath shaker for another 41 h. At the beginning the pH was set to 6.7 with 140 g/l KOH and then the co-cultivation was carried out without pH control.

During cultivation several 5 mL samples were taken to determinate OD₆₀₀ₙₘ, pH and product formation. The determination of the product concentrations is performed by LCMS and semiquantitative ¹H-NMR spectroscopy. As an internal quantification standard sodium trimethylsilylpropionate (T(M)SP) was used.

During the co-cultivation the concentration of acetate increased from 0.79 g/L to 1.83 g/L, for propionate from 0.00 g/L to 0.23 g/L, for propanol from 0 to 19 mg/L, for butyrate from 0 to 14 mg/L, and for formate from 32 mg/L to 335 mg/L. The concentration of ethanol decreased from 47 mg/L to 25 mg/L during this time.

### Example 6

Production and Processing of Propanol from Synthesis Gas with Clostridium autoethanogenum and Clostridium neopropionicum

For the biotransformation of hydrogen and carbon dioxide to propanol the homoacetogenic bacterium Clostridium autoethanogenum was cultivated on synthesis gas with a subsequent cultivation step with Clostridium neopropionicum. All cultivation steps were carried out under anaerobic conditions in pressure-resistant glass bottles that can be closed airtight with a butyl rubber stopper. Afterwards the product was extracted with a mixture of polypropylene glycol and hexadecane (70/30% w/w) or oleyl alcohol in two separate extractions.

For the cultivation of C. autoethanogenum 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂)×2H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO4×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock. The chemolithoautotrophic cultivation was carried out in 1 L pressure-resistant glass bottle at 37° C., 100 rpm and a ventilation rate of 3 L/h with a premixed gas with 67% H₂ and 33% CO₂ in an open water bath shaker for 73 h till OD₆₀₀ₙₘ of 0.39. The gas was discharged into the medium through a sparger with a pore size of 10 μm, which was mounted in the center of the reactors. Then the cell suspension was centrifuged.

For the main culture of C. autoethanogenum 500 ml LM33 mineral medium (pH=5.8, 1.3 g/L KOH, 0.5 g/L MgCl₂, 0.21 g/L NaCl, 0.135 g/L CaCl₂)×2H₂O, 2.65 g/L NaH₂PO₄×2H₂O, 0.5 g/L KCl, 2.5 g/L NH₄Cl, 15 mg/L nitrilotriacetic acid, 30 mg/L MgSO₄×7 H₂O, 5 mg/L MnSO₄×H₂O, 1 mg/L FeSO₄×7 H₂O, 8 mg/L Fe(SO₄)₂(NH₄)₂×6 H₂O, 2 mg/L CoCl₂×6 H₂O, 2 mg/L ZnSO₄×7 H₂O, 200 μg/L CuCl₂×2 H₂O, 200 μg/L KAI(SO₄)₂×12 H₂O, 3 mg/L H₃BO₃, 300 μg/L Na₂MoO₄×2 H₂O, 200 μg/L Na₂SeO₃, 200 μg/L NiCl₂×6 H₂O, 200 μg/L Na₂WO4×6 H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B12; 500 μg/L p-aminobenzoate; 500 μg/L lipoic acid, 10 mg/L FeCl₃, aerated for 30 min with a premixed gas with 67% H₂ and 33% CO₂, with additional 500 mg/L L-cysteine-hydrochlorid) were inoculated with cells from the first preculture to an OD₆₀₀ₙₘ of 0.17. The chemolithoautotrophic cultivation was carried out in a 1 L pressure-resistant glass bottle at 37° C., 150 rpm, with automatic pH control at pH 5.5 and a ventilation rate of 1 L/h with a premixed gas with 67% H₂, 33% CO₂ in an open water bath shaker for 187 h. The gas was discharged into the medium through a sparger with a pore size of 10 μm, which was mounted in the center of the reactors. After cultivation the medium was sterile-filtered for the next step.

During 187 h 4.6 g/L ethanol and 14.6 g/L acetate were produced.

For the first preculture of C. neopropionicum 100 ml DSMZ318 medium (pH 6.7; 0.61 g/l NaCl, 0.047 g/l MgCl₂, 0.30 g/l KH₂PO₄, 1.00 g/l NH₄Cl, 0.081 g/l CaCl₂)×2H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl₃×6 H₂O, 1 mg/L MnCl₂×4 H₂O, 0.24 mg/L CoCl₂×6 H₂O, 1 mg/L ZnCl₂, 0.25 mg/L CuCl₂×2 H₂O, 0.1 mg/L H₃B03, 0.24 mg/L Na₂MoO₄×2 H₂O, 1.2 mg/L NiCl₂×6 H₂O, 0.26 mg/L Na₂SeO₃×5 H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B12, 0.05 mg/L p-aminobenzoate, 0.05 mg/L lipoic acid, 0.25 g/L cysteine-HCl×H₂O) in a 250 ml bottle were inoculated with 5 ml of a frozen cryoculture and flushed with a premixed gas with 67% H₂, 33% CO₂ to an overpressure of 0.8 bar. The culture was incubated at 30° C. and 100 rpm in an open water bath shaker for 20 h till an OD₆₀₀ₙₘ>0.12. Then the cell suspension was centrifuged.

For a second preculture of Clostridium neopropionicum 200 mL of fresh DSMZ318 medium in a 500 mL bottle were inoculated with centrifuged cells from the first preculture to an OD₆₀₀ₙₘ of 0.03 and flushed with a premixed gas with 67% H₂, 33% CO₂ to an overpressure of 0.8 bar. This growing culture was incubated at 30° C. and 100 rpm in an open water bath shaker for 25 h till an OD₆₀₀ₙₘ>0.2. Then the cell suspension was centrifuged.

For the main culture 150 mL of the sterile-filtered medium from the main culture of C. autoethanogenum were flushed first with nitrogen and afterwards with a premixed gas with 67% H₂, 33% CO₂ to an overpressure of 0.8 bar. 0.25 g/L L-cysteine-hydrochloride were added and the pH was set to 6.8 with NaOH. Half of the cells from the second preculture of C. neopropionicum, necessary for an OD₆₀₀ₙₘ of 0.1, were added to the medium. The cultivation was carried out in a 500 mL pressure-resistant glass bottle at 30° C., 100 rpm, with automatic pH control with NaOH at pH 6.8 in an open water bath shaker for another 88 h.

During the cultivation time 2 g/L ethanol were consumed and 2 g/L propionate and 49 mg/L propanol were produced.

During both cultivations several 5 mL samples were taken to determinate OD₆₀₀ₙₘ, pH and product formation. The determination of the product concentrations was performed by HPLC and NMR spectroscopy.

For extraction of propanol two 50 mL samples of the main culture with C. neopropionicum were shaken at 30° C. under anaerobic conditions in a separation funnel with extractant for 3 minutes and the two phases were separated afterwards with the separation funnel. As extractant 50 mL of oleyl alcohol (MS-295), 85%, Abcr GmbH) or a mixture of 70% w/w polypropylene glycol (MS-346, contains 130-190 ppm Aldrich) and 30% w/w hexadecane ((MS-303), 99%, Sigma), were used as two different extractants.

In both cases propanol was extracted completely into the organic phase.

### Example 7

### Extraction of Butanol

For the extraction of butanol, two separate extracting mediums were used (1) oleyl alcohol and (2) a mixture of polypropylene glycol and hexadecane (70/30% w/w) was used.

For the extraction with 70% w/w polypropylene and 30% w/w hexadecane, 50 g of a butanol solution with 0.522 g/kg were shaken at ambient temperature (20-25° C.) in a separation funnel with 46 g of the extracting medium for two minutes. The two phases were separated and butanol concentration in the aqueous phase was determined.

After extraction the butanol concentration in the aqueous phase had decreased to 0.251 g/kg. The distribution coefficient calculated by mass balance was 1.17.

For the extraction with oleyl alcohol 4.81 g of a butanol solution with 5.97 g/kg were shaken at ambient temperature (20-25° C.) in a centrifuge tube with 2.02 g extractant for 30 minutes. The two phases were separated and the concentration in the aqueous phase was determined.

After extraction butanol concentration in the aqueous phase had decreased to 2.40 g/kg. The distribution coefficient calculated by mass balance was 2.97.

1. A method of producing at least one alcohol from a carbon source, the method comprising:
(a) producing the alcohol in an aqueous medium under aerobic conditions; and
(b) extracting the alcohol from (a) from the aqueous medium by:
(bi) contacting the alcohol in the aqueous medium with at least one extracting medium for a time sufficient to extract the alcohol from the aqueous medium into the extracting medium, and
(bii) separating the extracting medium with the extracted alcohol from the aqueous medium,
wherein the extracting medium comprises oleyl alcohol,
the alcohol comprises at least 3 carbon atoms, and
the carbon source comprises CO and/or CO₂.

2. The method according to claim 1, wherein the alcohol is propanol and (a) comprises
(ai) contacting the carbon source with a reaction mixture comprising
a first acetogenic microorganism in an exponential growth phase;
free oxygen; and
a second acetogenic microorganism in a stationary phase,
wherein the first and second acetogenic microorganisms are capable of converting the carbon source to the acetate and/or ethanol; and
(aii) contacting the acetate and/or ethanol from (ai) with a third microorganism capable of converting the acetate and/or ethanol to the propanol in the aqueous medium.

3. The method according to claim 1, wherein the alcohol is at least one higher alcohol and (a) comprises
(a1) contacting the carbon source with a reaction mixture comprising
a first acetogenic microorganism in an exponential growth phase;
free oxygen; and
a second acetogenic microorganism in a stationary phase
wherein the first and second acetogenic microorganisms are capable of converting the carbon source to the acetate and/or ethanol;
(a2) contacting the acetate and/or ethanol from (a1) with a fourth microorganism capable of carrying out the ethanol carboxylate fermentation pathway and converting the acetate and/or ethanol from (a1) to form an acid; and
(a3) contacting the acid from (a2) with the reaction mixture of (a1),
wherein the first and/or second acetogenic microorganisms are capable of converting the acid to the corresponding higher alcohol in the aqueous medium, and
wherein the higher alcohol comprises 4 to 10 carbon atoms.

4. The method according to claim 2, wherein the first and second microorganisms are each independently selected from the group consisting of Clostridium autothenogenum DSMZ 19630, Clostridium ragsdahlei ATCC no. BAA-622, Clostridium autoethanogenum, Moorella sp HUC22-1, Moorella thermoaceticum, Moorella thermoantotrophica, Rumicoccus productus, Acetoanaerobum, Oxobacter pfennigii, Methanosarcina barkeri, Methanosarcina acetivorans, Carboxydothermus, Desulfotomaculum kutznetsovii, Pyrococcus, Peptostreptococcus, Butyribacterium methylotrophicum ATCC 33266, Clostridium formicoaceticum, Clostridium butyricum, Lactobacillus delbrukii, Propionibacterium acidoproprionici, Proprionispera arboris, Anaerobierspirillum succiniproducens, Bacterioides amylophilus, Becterioides ruminicola, Thermoanaerobacter kivui, Acetobacterium woodii, Acetoanaerobium notera, Clostridium aceficum, Butyribacterium methylotrophicum, Moorell athermoacetica, Eubacterium limosum, Peptostreptococcus productus, Clostridium ljungdahlii, Clostridium ATCC 29797 and Clostridium carboxidivorans.

5. The method according to claim 2, wherein the first acetogenic microorganism in the exponential growth phase has a growth rate of 0.01 to 2 h⁻¹.

6. The method according to claim 1, wherein the aerobic conditions is a result of oxygen being at a concentration of 0.000005 to 1% volume in the gas phase.

7. The method according to claim 2, wherein the third microorganism is a propionogen.

8. The method according to claim 7, wherein the third microorganism is a genetically modified organism comprising increased expression relative to the wild type cell of propionate CoA-transferase (E₁), lactoyl-CoA dehydratase (E₂) and acryloyl-CoA reductase (E₃).

9. The method according to claim 7, wherein the third microorganism is Clostridium neopropionicum.

10. The method according to claim 2, wherein the first and second microorganism is microorganisms are Clostridium autoethanogenum and the third microorganism is Clostridium neopropionicum.

11. The method according to claim 3, wherein the fourth microorganism is selected from the group consisting of Clostridium kluyveri, and C. Carboxidivorans.

12. The method according to claim 1, wherein (a) and (b) are carried out in a single fermenter.

13. The method according to claim 3, wherein the higher alcohol is at least one selected from the group consisting of 1-butanol, 2-methyl-1-butanol, isobutanol, 3-methyl-1-butanol, 1-hexanol, 1-octanol, 1-pentanol, 1-heptanol, 3-methyl-1-pentanol, 4-methyl-1-hexanol, 5-methyl-1-heptanol, 4-methyl-1-pentanol, 5-methyl-1-hexanol, and 6-methyl-1-heptanol and combinations thereof.
